SEQUENCE LISTING (2 sequences)

SEQ ID NO: 1 - DNA, 21 bp, Artificial sequence
  primer
agctctgcatcgttttgggtt

SEQ ID NO: 2 - DNA, 24 bp, Artificial sequence
  primer
gttccattatccgctacatctgaa

(19) United States
(12) Patent Application Publication
(10) Pub. No.: US 2017/0058353 A1
(43) Pub. Date: Mar. 2, 2017

(54) QPCR-BASED METHOD TO ASSESS T CELL FUNCTION

(71) Applicant: Tonya J Webb, Glen Burnie, MD (US)

(72) Inventor: Tonya J Webb, Glen Burnie, MD (US)

(21) Appl. No.: 15/118,852

(22) PCT Filed: Feb. 12, 2015
(86) PCT No.: PCT/US15/15685

§ 371 Date: Aug. 12, 2016

Related U.S. Application Data
(60) Provisional application No. 61/938,743, filed on Feb. 12, 2014.

(51) Int. Cl. C12Q 1/68

(52) U.S. Cl. CPC C12Q 1/6886; C12Q 2600/158

(57) ABSTRACT

The invention provides a method of determining T cell function in a subject in need of immunotherapy comprising:
i) providing a blood sample comprising a population of T cells from the subject;
ii) activating the T cells in the sample; and
iii) assaying an expression level of one or more T cell activation markers using quantitative real time PCR (qPCR) after activating the T cells in the sample.

### CROSS REFERENCE TO RELATED APPLICATIONS

This application claims the benefit of U.S. Provisional Appl. No. 61/938,743, filed Feb. 12, 2014. The content of the aforesaid application is relied upon and incorporated by reference in its entirety.

### STATEMENT OF FEDERALLY SPONSORED RESEARCH AND DEVELOPMENT

This invention was made with government support under Grant No. CA162273 awarded by the National Institute of Health. The government has certain rights in the invention.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 611 Byte ASCII (Text) file named “seq_listing.txt,” created on Feb. 12, 2015.

### FIELD OF THE INVENTION

The present invention generally relates at least to the fields of molecular biology, immunology, cancer biology and medicine. In particular, the invention relates to a sensitive method for assessing T cell and Natural Killer T (NKT) cell function from a small amount of patient blood sample.

### BACKGROUND

Immunotherapy (sometimes called biological therapy, biotherapy, or biological response modifier therapy) uses the body's immune system, either directly or indirectly, to treat ailments or diseases, including cancer. Immunotherapy is often used as an adjunct to conventional therapies. Immunotherapeutic strategies include administration of vaccines, activated cells, antibodies, cytokines, chemokines, as well as small molecular inhibitors, anti-sense oligonucleotides, and gene therapy (Mocellin et al., Cancer Immunol. & Immunother. 51: 583-595 (2002); Dy et al., J. Clin. Oncol. 20: 2881-2894 (2002)).

The aim of any immunotherapeutic treatment is to minimize nonspecific toxicity, thus utilizing the body's own mechanisms to target and kill abnormal cells. Immunotherapy can be used to prime and amplify antigen-specific lymphocytes either in vivo (active iummunotherapy) or ex vivo prior to their infusion (adoptive immunotherapy). Adoptive immunotherapy is a procedure whereby an individual's own lymphocytes can be expanded ex vivo and re-infused back into the body. Both adoptive and active immunotherapy can be used as therapeutic strategies for the treatment of viral infection (Papadopoulos et al., N. Engl. J. Med, 330(17):1185-91 (1994); Savoldo et al., Leuk Lymphoma, 39(5-6):455-64 (2000)), autoimmune disease (Hori et al., Adv. Immunol., 81:331-71 (2003); Karim et al., J. Immunol., 172(2):923-8 (2004)), and cancer (Dudley, et al., Nat. Rev. Cancer, 3(9):666-75 (2003); Riddell et al., Cancer Control, 9(2):114-22 (2002); Yee et al., Proc. Natl. Acad. Sci. USA., 99(25):16168-73 (2002)).

The concept of adoptive cellular therapy for tumors has at its goal the elimination of cancer through the transfer of activated T-cells and/or natural killer cells. In general, to prime the cells, peripheral T-cells can be removed from the patient, activated ex vivo, and then re-infused. The step of ex vivo activation can also include exposure to the patient's tumor cells or to a tumor cell vaccine.

Natural killer T (NKT) cells recognize lipid antigen presented in the context of the non-classical major histocompatibility class I molecule, CD1d (Dellabona et al., J Exp Med 180, 1171-6 (1994); Lantz et al., J Exp Med 180, 1097-106 (1994); Berzins et al., Nat Rev Immunol 11, 131-42 (2011); Brennan et al., Nat Rev Immunol 13, 101-17 (2013)). Upon activation, NKT cells produce high amounts of cytokines which can stimulate other immune cells and initiate both innate and adaptive immune responses. While NKT cells comprise a relatively small percentage of lymphocytes (1-2% of mouse splenocytes and 0.01-2% of human peripheral blood mononuclear cells), they have been demonstrated to play important roles in autoimmune disease (Illes et al., J Immunol 164, 4375-81 (2000)), tumor surveillance (Terabe et al., Advances in cancer research 101, 277-348 (2008); Swann et al., Blood 113, 6382-5 (2009)), hematological cancers (Neparidze et al., Annals of the New York Academy of Sciences 1174, 61-7 (2009)), infectious disease and inflammatory conditions such as ischemia reperfusion injury (Kinjo et al., Nature 434, 520-5 (2005)). These effects are mediated through two defined subsets of NKT cells. Type I NKT cells (also known as invariant NKT cells, or iNKT cells) express an invariant Vα14Jα18 TCR in mice and Vα24Jα18 TCR in humans. Type II NKT cells are CD1d restricted T Cells that express a more diverse set of a chains in their TCR. The two types of NKT cells often exert opposing effects especially in tumor immunity where Type II cells generally suppress tumor immunity while Type I NKT cells enhance anti-tumor immune responses (Ambrosino et al., J Immunol 179, 5126-36 (2007)). In the present application, the focus on Type I NKT cells.

α-Galactosylceramide (α-GalCer) is a potent activator of iNKT cells (Burdin et al., J Immunol 161, 3271-3281 (1998); Carnaud et al., J Immunol 163, 4647-4650 (1999); Fujii et al., J Exp Med 198, 267-79 (2003); Hermans et al., J Immunol 171, 5140-7 (2003); Seino et al., Springer Semin Immunopathol 27, 65-74 (2005)). It was discovered during a screen for anti-tumor reagents derived from the marine sponge Agelas mauritianus (Kawano et al., Proc Natl Acad Sci USA 95, 5690-5693 (1998)). Now α-GalCer is the most extensively utilized and best-characterized antigen used to study NKT cell activation. Following stimulation with antigen presenting cells pulsed with α-GalCer, NKT cells produce T helper 1 (Th₁), Th₂ and Th₁₇ type cytokines (Cerundolo et al., Semin Immunol 22, 59-60 (2010); Monteiro et al., Crit Rev Immunol 34, 81-90 (2014); Singh et al., Hum Immunol 75, 250-260 (2014)). Because NKT cells can activate different immune cells and produce high amounts of immune cell stimulating cytokines like interferon-γ (IFN-γ), interleukin-4 (IL-4) and IL-10 they are considered an important immunoregulatory cell type that plays a pivotal role in modulating the immune responses (Godfrey et al., Nat Rev Immunol 4, 231-7 (2004); Sun et al., J Interferon Cytokine Res 32, 505-16 (2012)).

Given that NKT cells can mediate potent anti-tumor immune responses, they have been considered as a novel immunotherapeutic target (Mattarollo et al., Int J Cancer 119, 1630-7 (2006); Fujii, S., Trends Immunol 29, 242-9 (2008)). In two studies, patients with advanced cancers were injected with either α-GalCer (Giaccone et al., Clin Cancer Res 8, 3702-9 (2002)) or α-GalCer loaded immature dendritic cells (Nieda et al., Blood 103, 383-9 (2004)) in order to modulate NKT cell responses. Chang and colleagues showed that multiple injections of α-GalCer loaded mature dendritic cells lead to sustained expansion of NKT cells and antigen specific T cells (Chang et al., J Exp Med 201, 1503-17 (2005)). However, these expanded NKT cells from cancer patients still exhibited reduced capacity for IFN-γ secretion compared to NKT cells from healthy controls. Recent clinical trials evaluating the effectiveness of NKT cell based immunotherapy in treating patients with solid tumors in the liver or lung, as well as unresectable head and neck cancers, have shown some promise (Ishikawa et al., Clin Cancer Res 11, 1910-7 (2005); Uchida et al., Cancer Immunol Immunother 57, 337-45 (2008)). Collectively, these studies and others (Kawano et al., Cancer Res 59, 5102-5105 (1999); Tahir et al., J Immunol 167, 4046-50 (2001); Fujii et al., Br J Haematol 122, 617-22 (2003)) have demonstrated that many cancer patients have a deficiency in both NKT cell number and function, which suggests that in vivo NKT cell modulation would only be effective in patients with sufficient numbers of functional NKT cells.

Several groups have shown that soluble forms of CD1d molecules loaded with lipid antigen are directly able to target NKT cells in vitro (Naidenko et al., J Exp Med 190, 1069-1080 (1999); Schumann et al., J Immunol 170, 5815-5819 (2003); Sriram et al., Eur J Immunol 35, 1692-701 (2005)), and this has been used to develop a method to ex vivo activate and expand NKT cells using CD1D-based artificial antigen presenting cells (aAPC) (Webb et al., J Immunol Methods 346, 38-44 (2009); East et al., J Vis Exp (70):pii: 4333. doi: 10.3791/4333 (2012); Sun et al., J Interferon Cytokine Res 32, 505-16 (2012)). The beads are loaded with CD1d dimers that bind α-GalCer and anti-CD28 antibodies to also activate the costimulatory molecule on the cell surface of NKT cells. The complex of α-GalCer/CD1d binds to the NKT cell TCR.

The use of aAPC allows a rapid, reproducible, and standardized method to examine NKT cell function. NKT cell function is typically assessed by enzyme-linked immunosorbent assay (ELISA), enzyme-linked immunospot (ELISPOT) and intracellular staining (ICS). A disadvantage of these methods is that a large blood volume is needed in order to obtain a sufficient number of peripheral blood mononuclear cells (PBMC) to assess the activation of specific T cell subsets. To circumvent these issues, Ndhlovu et al. developed an assay to detect low-frequency measles virus-specific CD8⁺T cells in whole blood (Ndhlovu et al., Clin Vaccine Immunol 16, 1066-73 (2009)). This highly sensitive assay only requires a minimal amount of blood.

Accordingly, there is a need for improved methods for assaying T cell function in subjects that are rapid and sensitive, particularly in patients prior to undergoing immunotherapy, such as cancer patients. Herein, the inventor demonstrates that stimulation with CD1d-aAPC in combination with real time quantitative PCR (qPCR) can be used to rapidly assess the function of NKT cells within the peripheral blood.

### SUMMARY OF THE INVENTION

It is to be understood that both the foregoing general description of the embodiments and the following detailed description are exemplary, and thus do not restrict the scope of the embodiments.

According to non-limiting example embodiments, the invention provides a method of determining T cell function in a subject comprising:

i) providing a blood sample comprising a population of T cells from the subject;

ii) activating the T cells in the sample; and

iii) assaying an expression level of one or more T cell activation markers using quantitative real time PCR (qPCR) after activating the T cells in the sample.

In some embodiments, the subject is in need of immunotherapy for one or more diseases or conditions.

In another aspect, the invention provides a method for rapidly assessing the function of natural killer T (NKT) cells within the peripheral blood of an animal using quantitative real time PCR (qPCR).

In another embodiment, the invention provides a method for assessing baseline NKT cell function in a patient in need of immunotherapy comprising utilizing antigen presenting cells (APC) in combination with qPCR to determine which patients can benefit from NKT cell-based therapies.

In another embodiment, the invention provides a method for assessing baseline NKT cell function in a patient in need of immunotherapy comprising utilizing artificial antigen presenting cells (aAPC) in combination with qPCR by measuring the induction of different cytokines following stimulation to aid in determining which patients can benefit from NKT cell-based therapies.

In another embodiment, the invention provides a method for assessing baseline NKT cell function in a patient in need of immunotherapy comprising utilizing artificial antigen presenting cells (aAPC) in combination with qPCR by measuring the induction of gamma-interferon (IFN-γ) following stimulation to aid in determining which patients can benefit from NKT cell-based therapies.

In another embodiment, the invention provides a method for assessing baseline NKT cell function in a healthy individual comprising utilizing artificial antigen presenting cells (aAPC) in combination with qPCR by measuring the induction of gamma-interferon (IFN-γ) following stimulation with α-Galactosylceramide (α-GalCer).

In another embodiment, the invention provides a method for assessing baseline NKT cell function in a patient in need of immunotherapy comprising utilizing artificial antigen presenting cells (aAPC) in combination with qPCR by measuring the induction of different cytokines following stimulation with α-Galactosylceramide (α-GalCer) to aid in determining which patients can benefit from NKT cell-based therapies.

In another embodiment, the invention provides a method for assessing baseline NKT cell function in a patient in need of immunotherapy comprising utilizing artificial antigen presenting cells (aAPC) in combination with qPCR by measuring the induction of gamma-interferon (IFN-γ) following stimulation with α-Galactosylceramide (α-GalCer) to aid in determining which patients can benefit from NKT cell-based therapies.

In another embodiment, the invention provides a method for assessing baseline NKT cell function in a breast cancer patient in need of immunotherapy comprising utilizing artificial antigen presenting cells (aAPC) in combination with qPCR by measuring the induction of different cytokines following stimulation with α-Galactosylceramide (α-GalCer) to aid in determining which patients can benefit from NKT cell-based therapies.

In another embodiment, the invention provides a method for assessing baseline NKT cell function in a patient in need of immunotherapy comprising utilizing artificial antigen presenting cells (aAPC) in combination with qPCR by measuring the induction of different cytokines selected from the group consisting of GM-CSF, TNF-α, and IL-17A following stimulation to aid in determining which patients can benefit from NKT cell-based therapies.

In another embodiment, the invention provides a method for determining which patients can benefit from NKT cell-based immunotherapeutic strategy comprising utilizing artificial antigen presenting cells (aAPC) in combination with qPCR by measuring the induction of gamma-interferon (IFN-γ) following stimulation with α-Galactosylceramide (α-GalCer) wherein those patients with the higher baseline NKT cell function are the best candidates for NKT-cell based therapies.

In another embodiment, the invention provides a method for determining which breast cancer patients can benefit from NKT cell-based immunotherapeutic strategy comprising comprising utilizing artificial antigen presenting cells (aAPC) in combination with qPCR by measuring the induction of gamma-interferon (IFN-γ) following stimulation with α-Galactosylceramide (α-GalCer) wherein those patients with the higher baseline NKT cell function are the best candidates for NKT-cell based therapies.

In another embodiment, the invention provides a method for identifying and quantifying NKT cells comprising contacting a body fluid such as blood of an individual with a CD1D-based artificial antigen presenting cells (aAPC) which bind the NKT cells via the TCR and further assaying NKT cell function with quantitative PCR (qPCR).

In another embodiment, the present invention relates to a method for identifying and quantifying NKT cells comprising contacting a body fluid such as blood of an individual with a CD1D-based artificial antigen presenting cells (aAPC) which bind the NKT cells via the TCR and further assaying NKT cell function by measuring IFN-γ induction using quantitative real time PCR (qPCR).

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Circulating percentages of NKT cells are low in healthy donors and breast cancer patients. Peripheral blood mononuclear cells (PBMC) were collected from healthy donors and breast patients. Cells were stained for Vα24⁻Vβ11⁺ or iNKT (6B11)/CD3 to determine the percentage of NKT cells within the lymphocyte population and analyzed by FACS. (A) Representative dot plots are shown from healthy donors (HD) and breast cancer patients (BC). (B) Scatter plots demonstrate the variation in the percentages of NKT cells.

FIG. 2. PBMC produce multiple cytokines within hours following stimulation. (A) PBMC were stimulated with PMA and ionomycin for the indicated time period. Culture supernatants were harvested and standard sandwich ELISA was used to measure TNF-α, GM-CSF, or IFN-γ production. (B) Human T cells rapidly produce cytokines following ex vivo stimulation. PBMC were stimulated with anti-CD3/CD28 microbeads and cytokines were measured by ELISA. PBMC were stimulated with anti-CD3/CD28 microbeads for 0, 2 and 4 hours. This is a representative experiment of eight similar experiments.

FIG. 3. qPCR provides a sensitive method for detecting NKT cells in the peripheral blood. Healthy human NKT cells were added in increasing concentrations to donor PBMCs. The cells were stained with PE-conjugated CD3 and APC-conjugated α-GC tetramer antibodies, with unloaded tetramer antibody serving as a negative control. The cells were analyzed by flow cytometry and percentage of NKT cells was calculated by subtracting unloaded tetramer from α-GC tetramer staining. Loaded tetramer percentage is listed on the left y-axis, while Vα24 mRNA fold change by qPCR is shown on the right y-axis. The total NKT cell number is displayed on the x-axis. The data shown are the average of three independent experiments.

FIG. 4. Schematic diagram of experimental design. In this study, PBMC from healthy donors and breast cancer patients were stimulated with various stimuli: empty beads, CD1d-aAPC, anti-CD3/CD28 microbeads, or PMA and ionomycin for four hours. In this system, empty microbeads served as a negative control. To specifically activate NKT cells using aAPC, CD1d-Ig is used to provide the cognate antigen specific signal through the TCR (signal 1) and anti-CD28 mAb provides signal 2. These aAPC were pre-loaded with α-GalCer in order to induce cytokine production by classic type I NKT cells. Simulation with PMA and ionomycin was used as the positive control. Following stimulation, the supernatants were collected for ELISA and the RNA was extracted from the cell pellets and used for RT-PCR and qPCR.

FIG. 5. CD1d-based aAPC in combination with qPCR can be used to assess NKT cell activation. (A) A representative dot plot is shown from HD1 and a scatter plot indicates the % NKT cells from each of the donors shown in panels C-E. (B) Schematic of CD1d-based aAPC. Stimulation with α-GalCer loaded CD1d-Ig aAPC induces IFN-γ expression in NKT cells in healthy donor PBMC. PBMC were incubated for 4 h with either empty beads, CD1d-aAPC, anti-CD3/CD28 microbeads, or PMA/ionomycin. NKT cell activation was assessed by measuring IFN-γ (C) mRNA levels by RT-PCR (D) production in the supernatants by ELISA (E) induction by qPCR. RT-PCR data in (C) is from donor 1, the other panels show data from four different healthy donors. Data shown are from one experiment and are representative of 3 independent experiments.

FIG. 6. NKT cell function in breast cancer patients can be assessed using CD1D-based aAPC in combination with qPCR. (A) Flow cytometric analysis of circulating NKT cells from breast cancer (BC) patients shown in panels B-D. PBMC from breast cancer patients were incubated for 4 h with empty beads, CD1d-aAPC, anti-CD3/CD28 microbeads, or PMA/ionomycin. (B) NKT cell activation was assessed by measuring IFN-γ mRNA levels by RT-PCR (C) ELISA and (D) qPCR. RT-PCR data in (B) is from donor 3.

FIG. 7. NKT cells are reduced in cancer patients.

FIG. 8. Invariant NKT (iNKT) cells, NKT-like and NK cell percentages in Breast Cancer patients at GCC. NKT & NK populations are lower in AA, decrease with age and body weight.

FIG. 9. NKT cells can be expanded from some, but not all cancer patients.

### DETAILED DESCRIPTION

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of and/or “consisting of.” As used herein, the term “about” means at most plus or minus 10% of the numerical value of the number with which it is being used.

The present invention is based on the discovery of a sensitive method to rapidly assess T cell function in patients. In some embodiments, the method can be used to assay total T cell function. In other embodiments, NKT cell function can be assayed. The assay has particular utility in patients prior to undergoing immunotherapy. The assay also has particular relevance as a diagnostic or prognostic in various disease conditions where T cells or NKT cells are aberrantly activated. For example, in asthma, psoriasis, and atherosclerosis, NKT cells have been shown to be aberrantly activated. In some embodiments, the method could be used to detect the abnormal (over) activation of NKT cells and could be useful as a diagnostic or prognostic test.

In some embodiments, the methods can be performed prior to immunotherapy to assay baseline levels of T cell, and/or NKT cell activation in order to determine which patients may benefit from T cell-based therapies. NKT cells comprise a rare, but important subset of T cells which account for ˜0.2% of the total circulating T cell population. NKT cells are known to have anti-tumor functions and rapidly produce high levels of cytokines following activation. Several clinical trials have sought to exploit the effector functions of NKT cells. While some studies have shown promise, NKT cells can be approximately 50% lower or more in cancer patients compared to healthy donors of the same age and gender, thus limiting their therapeutic efficacy. Accordingly, it is currently difficult to determine which patients would benefit from T cell or NKT cell-based immunotherapy. These studies indicate that baseline levels of activation should be assessed before initiating an NKT cell based immunotherapeutic strategy, thus the goal of this study was to develop a sensitive method to rapidly assess NKT cell function. Artificial antigen presenting cells were utilized in combination with qPCR in order to determine NKT cell function in peripheral blood mononuclear cells from healthy donors and breast cancer patients. It was found that NKT cell activation can be detected by qPCR, but not by ELISA, in healthy donors as well as in breast cancer patients following four hour stimulation. This method utilizing CD1d-expressing aAPC can be used as a novel tool in adoptive immunotherapeutic strategies.

In some embodiments, the invention provides a method of determining T cell function in a subject comprising:

i) providing a blood sample comprising a population of T cells from the subject;

ii) activating the T cells in the sample; and

iii) assaying an expression level of one or more T cell activation markers using quantitative real time PCR (qPCR) after activating the T cells in the sample.

In some embodiments, the invention provides a method of determining NKT cell function in a subject in need of immunotherapy comprising:

i) providing a blood sample comprising a population of T cells from the subject;

ii) activating the NKT cells in the sample with an artificial antigen presenting cell comprising CD1d bound to α-GalCer; and

iii) assaying an expression level of one or more T cell activation markers using quantitative real time PCR (qPCR) after activating the T cells in the sample.

In some embodiments, the invention provides a method of determining total T cell function in a subject in need of immunotherapy comprising:

i) providing a blood sample comprising a population of T cells from the subject;

ii) activating the total T cells in the sample with an artificial antigen presenting cell comprising anti-CD3 and anti-CD28; and

iii) assaying an expression level of one or more T cell activation markers using quantitative real time PCR (qPCR) after activating the T cells in the sample.

In some embodiments, the invention provides a method of determining T cell function in a subject comprising:

i) providing a blood sample comprising a population of T cells from the subject;

ii) activating the T cells in the sample; and

iii) assaying an expression level of one or more T cell activation markers using quantitative real time PCR (qPCR) after activating the T cells in the sample, wherein the subject has a disease wherein T cells are aberrantly activated.

In some embodiments, the invention provides a method of determining NKT cell function in a subject comprising:

i) providing a blood sample comprising a population of T cells from the subject;

ii) activating the NKT cells in the sample; and

iii) assaying an expression level of one or more T cell activation markers using quantitative real time PCR (qPCR) after activating the T cells in the sample, wherein the subject has a disease wherein NKT cells are aberrantly activated. In some embodiments, the subject has a disease selected from the group consisting of asthma, psoriasis, and atherosclerosis.

The subject whose T cells will be activated and assayed is not limiting. In some embodiments, the subject is in need of immunotherapy for one or more diseases or conditions. In some embodiments, the subject has a disease or condition where T cells are aberrantly activated. In some embodiments, the subject is a normal, healthy subject. In some embodiments, the subject is a mammal. In some embodiments, the mammal is selected from the group consisting of a human, mouse, rat, guinea pig, cat, dog, horse, cow, sheep or pig.

In some embodiments, the expression level of the one or more T cell activation markers is compared between a subject in need of immunotherapy for one or more diseases or conditions and a normal, healthy subject.

In some embodiments, the expression level of the one or more T cell activation markers is compared between a subject having a disease in which T cells are aberrantly activated and a normal, healthy subject.

The blood sample comprising a population of T cells is not limiting. In some embodiments, the blood sample can include whole blood or fractionated blood comprising a population of T cells. In some embodiments, the blood sample comprising a population of T cells is an isolated peripheral blood mononuclear cell sample (PBMC). PBMC can be prepared using known methods and techniques. In some embodiments, PBMC can be isolated by Ficoll-Hypaque (Amersham Pharmacia Biotek, Uppsala, Sweden) density gradient centrifugation. In some embodiments, approximately 10⁶ PBMC cells are stimulated. In some embodiments, the T cells are stimulated from 2-8 hours at 37° C. In some embodiments, the T cells are stimulated for about 4 hrs at 37° C.

In some embodiments, NKT cells are activated and the expression level of one or more NKT cell activation markers is assayed. NKT cells can recognize lipid or glycolipid antigen in the context of CD1d molecules and subsequently produce cytokines that activate cells of both the innate and adaptive immune responses. In some embodiments, the NKT cells are activated by CD1d bound to a lipid or glycolipid ligand. In some embodiments, the NKT cells are activated with CD1d bound to ligand on present on antigen presenting cells. Antigen presenting cells can include various cell types including cells as dendritic cells and macrophages. In some embodiments, the antigen presenting cells are the subjects own dendritic cells.

The CD1d bound ligand is not limiting provided it is able to activate NKT cells when to CD1d. In some embodiments, the CD1d bound ligand is selected from the group consisting of C-glycosidific form of alpha-galactosylceramide (α-C-GalCer, alpha-galactosylceramide (α-GalCer), 12 carbon acyl form of galactosylceramide (β-GalCer (C12)), β-D-glucopyranosylceramide (β-GlcCer), 1,2-diacyl-3-O-galactosyl-sn-glycerol (BbGL-II), diacylglycerol containing glycolipids (Glc-DAG-s2), Ganglioside (GD3), gangliotriaosylceramide (Gg3Cer), glycosylphosphatidylinositol (GPI), alpha-glucuronosylceramide (GSL-1), alpha-glucuronosylceramide (GSL-4), house dust extract+ovalbumin (HDE+OVA), isoglobotrihexosylceramide (iGb3), lipophosphoglycan (LPG), lyosphosphatidylcholine (LPC), alpha-galactosylceramide analog (OCH), phosphatidylcholine (PC), phosphatidylethanolamine (PE), phosphatidylinositol (PI), PI dimannoside (PIM4), phenyl pentamethyldihydrobenzofuran sulfonates (PPBF), sulfatide, phosphatidylserine (PS), threitolceramide and combinations thereof.

In some embodiments, the NKT cells are stimulated with an antigen presenting cell comprising CD1d. In some embodiments, the NKT cells are stimulated with an antigen presenting cell comprising CD1d bound to α-GalCer. In some embodiments, the NKT cells are stimulated with an artificial antigen presenting cell comprising CD1d. In some embodiments, the NKT cells are stimulated with an artificial antigen presenting cell comprising CD1d bound to α-GalCer. In some embodiments, the artificial antigen presenting cell comprises a magnetic bead loaded with CD1d bound to ligand.

In some embodiments, the ligand is added directly to the blood sample where it will become associated with endogenous CD1d present on cells in the sample and thereby activate NKT cells. In some embodiments, the ligand is combined with antigen presenting cells prior to addition to the blood sample. In some embodiments, the ligand and antigen presenting cells are added separately to the blood sample.

In some embodiments, artificial antigen presenting cells (aAPC) are used to activate total T cells or NKT cells. In some embodiments, where NKT cells are activated, the aAPC comprise CD1d loaded beads which, when combined with a ligand to bind CD1d, are able to bind to NKT cells and induce their activation. In some embodiments, the CD1D-based aAPC can be prepared as described in Shiratsuchi et al., J Immunol Methods 345, 49-59 (2009) or Webb et al., J Immunol Methods 346, 38-44 (2009) which are incorporated herein by reference. In some embodiments of making the aAPC for activating NKT cells, hCD1d-Ig (Pharmingen) can be added to epoxy beads (Dynal, product #140.01, Dynabeads, M-450, Epoxy, 4×10⁸ beads/ml) in sterile 0.1M Borate buffer, pH 7.0-7.4, in the presence of anti-CD28 mAb (Biolegend). The bead protein combination can be mixed with rotation and incubated for 24 h at 4° C. The beads can be subsequently washed and the hCD1d molecules loaded with 40× molar excess lipid antigen, such as α-GalCer (Enzo) in PBS, calculated based on the amount of hCD1d-Ig protein added to the beads.

In some embodiments, the total T cells are stimulated with an antigen presenting cell. In some embodiments, the total T cells are stimulated with an artificial antigen presenting cell. In some embodiments, where total T cells are activated, anti-CD3 and anti-CD28 are added to microbeads for use as artificial antigen presenting cells. In some embodiments, about 20 μg of each mAb (Biolegend) can be added to 4×10⁸ beads.

In some embodiments, the one or more T or NKT cell activation markers is selected from the group consisting of IFN-γ, TNF-α, and GM-CSF. In one embodiment, the marker to be assayed is IFN-γ. In one embodiment, IFN-γ can be assayed using the following primer sequences:
Forward primer AGCTCTGCATCGTTTTGGGTT (SEQ ID NO:1)
Reverse primer GTTCCATTATCCGCTACATCTGAA (SEQ ID NO:2)
   Assaying with these primers will give a product length of 118 base pair nucleic acid fragment.

In some embodiments, the subject is afflicted with a disease, such as cancer. In some embodiments, the subject has a cancer selected from the group consisting of breast cancer; bladder cancer; lung cancer; prostate cancer; thyroid cancer; leukaemia, lymphoma, CLL (chronic lymphocytic leukemia), CML (chronic myelocytic leukaemia), ALL (acute lymphoblastic leukaemia), AML (acute myelocytic leukaemia), PML (pro-myelocytic leukaemia), T-cell lymphoma, colon cancer; glioma; seminoma; liver cancer; pancreatic cancer; bladder cancer; renal cancer; cervical cancer; testicular cancer; head and neck cancer; ovarian cancer; neuroblastoma and melanoma.

In some embodiments, the subject has a bacterial infection, a viral infection, or a parasitic infection. In some embodiments, the infection is a bacterial infection from a bacteria selected from the group consisting of Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracia, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospria spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., and Rickettsia spp.

In some embodiments, the subject has a parasitic infection. In some embodiments, the parasitic infection is selected from the group consisting of amebiasis from Entamoeba histolytica, amebic meningoencephalitis from the genus Naegleria or Acanthamoeba, malaria from Plasmodium vivax, Plasmodium ovale, Plasmodium malariae, and Plasmodium falciparum, leishmaniasis from protozoa Leishmania donovani, Leishmania infantum, Leishmania chagasi, Leishmania tropica, Leishmania major, Leishmania aethiopica, Leishmania mexicana, and Leishmania braziliensis, Chagas' disease from the protozoan Trypanosoma cruzi, sleeping sickness from Trypanosoma brucei, Trypanosoma gambiense, and Trypanosoma rhodesiense, toxoplasmosis from Toxoplasma gondii, giardiasis from Giardia lamblia, cryptosporidiosis from Cryptosporidium parvum, trichomoniasis from Trichomonas vaginalis, Trichomonas tenax, Trichomonas hominis, pneumocystis pneumonia from Pneumocystis carinii, bambesosis from Bambesia microti, Bambesia divergens, and Bambesia bovis.

In some embodiments, the subject has a helminthic infection, including from the species: Taenia solium, Taenia saginata, Diphyllobothrium lata, Echinococcus granulosus, Echinococcus multilocularis, Hymenolepis nana, Schistosoma mansomi, Schistosoma japonicum, Schistosoma hematobium, Clonorchis sinensis, Paragonimus westermani, Fasciola hepatica, Fasciolopsis buski, Heterophyes heterophyes, Enterobius vermicularis, Trichuris trichiura, Ascaris lumbricoides, Ancylostoma duodenale, Necator americanus, Strongyloides stercoralis, Trichinella spiralis, Wuchereria bancrofti, Onchocerca volvulus, Loa loa, and Dracunculus medinensis.

In some embodiments, the subject has an infection from a fungal pathogen such as: Sporothrix schenckii, Coccidioides immitis, Histoplasma capsulatum, Blastomyces dermatitidis, Paracoccidioides brasiliensis, Candida albicans, Cryptococcus neoformans, Aspergillus fumigatus, Aspergillus flavus, fungi of the genera Mucor and Rhizopus, Fusarium solani and species causing chromomycosis such as those of the genera Phialophora and Cladosporium.

In some embodiments, the subject has an infection from a veterinary protozoal pathogen such as: Babesia caballi, Babesia canis, Babesia equi, Babesia felis, Balantidium coli, Besnoitia darlingi, Eimeria acervulina, Eimeria adenoeides, Eimeria ahata, Eimeria alabamensis, Eimeria auburnensis, Eimeria bovis, Eimeria brasiliensis, Eimeria brunetti, Eimeria canadensis, Eimeria cerdonis, Eimeria crandallis, Eimeria cylindrica, Eimeria debliecki, Eimeria despersa, Eimeria ellipsoidalis, Eimeria fauvei, Eimeria gallopavonis, Eimeria gilruthi, Eimeria granulosa, Eimeria hagani, Eimeria illinoisensis, Eimeria innocua, Eimeria intricate, Eimeria leuskarti, Eimeria maxima, Eimeria meleagridis, Eimeria meleagrimitis, Eimeria mitis, Eimeria mivati, Eimeria necatrix, Eimeria neodebliecki, Eimeria ninakohlyakimorae, Eimeria ovina, Eimeria pallida, Eimeria parva, Eimeria perminuta, Eimeria porci, Eimeria praecox, Eimeria punctata, Eimeria scabs, Eimeria spinoza, Zimeria subrotunda, Eimeria subsherica, Eimeria suis, Eimeria tenella, Eimeria wyomingensis, Eimeria zuernii, Endolimax gregariniformis, Endolimax nana, Entamoeba bovis, Entamoeba gallinarum, Entamoeba histolytica, Entamoeba suis, Giardia bovis, Giardia canis, Giardia cati, Giardia lamblia, Haemoproteus meleagridis, Hexamita meleagridis, Histomonas meleagridis, Iodamoeba buetschili, Isospora bahiensis, Isospora burrowsi, Isospora canis, Isospora fells, Isospora ohioensis, Isospora rivolta, Isospora suis, Klossiella equi, Leucocytozoon caallergi, Leucocytozoon smithi, Parahistomonas wenrichi, Pentatrichomonas hominis, Sarcocystis betrami, Sarcocystis bigemina, Sarcocystis cruzi, Sarcocystis fayevi, hemionilatrantis, Sarcocystis hirsuta, Sarcocystis miescheviana, Sarcocystis muris, Sarcocystis ovicanis, Sarcocystis tenella, Tetratrichomonas buttreyi, Tetratrichomonas gallinarum, Theileria mutans, Toxoplasma gondii, Toxoplasma hammondi, Trichomonas canistomae, Trichomonas gallinae, Trichomonas felistomae, Trichomonas eberthi, Trichomonas equi, Trichomonas foetus, Trichomonas ovis, Trichomonas rotunda, Trichomonas suis, and Trypanosoma melophagium.

In some embodiments, the subject has a viral infection. In some embodiments, the infection is caused by any one of a member of the Adenoviridae family, a member of the Coronavirus family, a member of the Picornaviridae family, a member of the Herpesviridae family, a member of the Hepadnaviridae family, a member of the Flaviviridae family, a member of the Retroviridae family, a member of the Orthomyxoviridae family, a member of the Paramyxoviridae family, a member of the Papovaviridae family, a member of the Rhabdoviridae family, or a member of the Togaviridae family.

In some embodiments, the method comprises assaying an expression level of one or more T cell activation markers using quantitative real time PCR (qPCR) from control T cells that have not been activated and comparing the expression level of one or more T cell activation markers from the activated with the expression level of one or more T cell activation markers from control T cells. In some embodiments, the activated T cells and the control T cells are from the same subject.

In some embodiments, the method comprises comparing the expression level of one or more T cell activation markers from the activated T cells from the subject in need of immunotherapy with the expression level of one or more T cell activation markers from activated T cells from a control, healthy subject.

In some embodiments, the methods further comprise administering to the subject an effective amount of immunotherapy. In some embodiments, if the expression level of the one or more T cell activation markers from the activated T cells from the subject in need of immunotherapy is at least 20% of the expression level of one or more T cell activation markers from activated T cells from a control, healthy subject, then the subject in need of immunotherapy is administered an effective amount of immunotherapy.

In some embodiments, if the expression level is at least 30%, at least 40%, at least 50%, at least 60%, at least 70%, at least 80% or at least 90% of the expression level of one or more T cell activation markers from activated T cells from a control, healthy subject, then the subject in need of immunotherapy is administered an effective amount of immunotherapy.

In some embodiments, the subject is not administered an immunotherapy when the expression level of the one or more T cell activation markers from the activated T cells from the subject in need of immunotherapy is below 10% of the expression level of one or more T cell activation markers from activated T cells from a control, healthy subject.

In some embodiments, the one or more T cell activation markers is induced at least 5-fold over cells that have not been activated. In some embodiments, the one or more T cell activation markers is induced at least 10-fold, at least 15-fold, at least 20-fold, at least 30-fold, at least 40-fold, and at least 50-fold over cells that have not been activated.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

Other embodiments of the invention are discussed throughout this application. Any embodiment discussed with respect to one aspect applies to other aspects as well and vice versa. Each embodiment described herein is understood to be embodiments that are applicable to all aspects of the invention. It is contemplated that any embodiment discussed herein can be implemented with respect to any device, method, or composition, and vice versa. Furthermore, systems, compositions, and kits of the invention can be used to achieve methods of the invention.

Aspects of the present teachings may be further understood in light of the following examples, which should not be construed as limiting the scope of the present teachings in any way.

### EXAMPLES

### Example 1

Development of a qPCR Method to Rapidly Assess the Function of NKT Cells

NKT cells comprise a rare, but important subset of T cells which account for ˜0.2% of the total circulating T cell population. NKT cells are known to have anti-tumor functions and rapidly produce high levels of cytokines following activation. Several clinical trials have sought to exploit the effector functions of NKT cells. While some studies have shown promise, NKT cells are approximately 50% lower in cancer patients compared to healthy donors of the same age and gender, thus limiting their therapeutic efficacy. These studies indicate that baseline levels of activation should be assessed before initiating an NKT cell based immunotherapeutic strategy, thus the goal of this study was to develop a sensitive method to rapidly assess NKT cell function. We utilized artificial antigen presenting cells in combination with qPCR in order to determine NKT cell function in peripheral blood mononuclear cells from healthy donors and breast cancer patients. We found that NKT cell activation can be detected by qPCR, but not by ELISA, in healthy donors as well as in breast cancer patients following four hour stimulation. This method utilizing CD1d-expressing aAPC will enhance our knowledge of NKT cell biology and could potentially be used as a novel tool in adoptive immunotherapeutic strategies.

### Materials and Methods

### Peripheral Blood Mononuclear Cells (PBMC)

PBMC were isolated by Ficoll-Hypaque (Amersham Pharmacia Biotek, Uppsala, Sweden) density gradient centrifugation or with BD Vacutainer PPT Tubes for Molecular Diagnostics (20-959-51D; Fisher Scientific, Suwanee, Ga.) All donors gave written informed consent before enrolling in the study. The Institutional Review Board at the University of Maryland School of Medicine approved this investigation. To optimize the protocol, leukocyte paks were purchased from a commercial vendor, Biological Specialty Corp., Colmar, Pa. The percentages of NKT cells were assessed in newly diagnosed patients, prior to treatment and healthy donors.

Preparation of Artificial Antigen Presenting Cells (aAPC)

CD1D-based aAPC were prepared as previously described (Shiratsuchi, T., Schneck, J., Kawamura, A. and Tsuji, M., 2009, Human CD1 dimeric proteins as indispensable tools for research on CD1-binding lipids and CD1-restricted T cells. J Immunol Methods 345, 49-59; Webb, T. J., Bieler, J. G., Schneck, J. P. and Oelke, M., 2009, Ex vivo induction and expansion of natural killer T cells by CD1d1-Ig coated artificial antigen presenting cells. J Immunol Methods 346, 38-44). In brief, to conjugate hCD1d-Ig dimer molecules to beads, 50 μg of hCD1d-Ig (Pharmingen) was added to 0.5 ml of epoxy beads (Dynal, product #140.01, Dynabeads, M-450, Epoxy, 4×10⁸ beads/ml) in sterile 0.1M Borate buffer, pH 7.0-7.4, in the presence of anti-CD28 mAb (Biolegend). The bead protein combination was mixed with rotation and incubated for 24 h at 4° C. The beads were subsequently washed and the hCD1D molecules were loaded with 40× molar excess lipid antigen (α-GalCer, Enzo) in PBS, calculated based on the amount of hCD1d-Ig protein added to the beads. For anti-CD3/CD28 microbeads, 20 μg of each mAb (Biolegend) was added to 4×10⁸ beads. The beads were washed and used as described above.

### Stimulation of PBMC

Human PBMC were cultured in complete medium: RPMI 1640 medium supplemented with non-essential amino acids (Sigma-Aldrich), sodium pyruvate (Gibco, Invitrogen Corporation), vitamin solution (Gibco), 2-mercaptoethanol (Gibco), 10% fetal calf serum (Gibco), and Pen/Strep (Gibco). PBMC (10⁶) were added to borosilicate glass vials (Wheaton Science Products) and different stimuli were added: empty beads as a negative control, anti CD3/CD28 beads, CD1d/CD28 beads and PMA (50 ng/ml) and ionomycin (1 μM) was used as a positive control. The total volume of cell culture was 1 ml and a 1:1 (PBMC: beads) ratio was maintained for each experiment.

For the time course studies, the PBMCs stimulated for 0, 30, 60, 120 or 240 minutes at 37° C. After the incubation the beads were removed via an EasySep magnet (Invitrogen) and the cells were transferred into 1.5 ml eppendorf tubes and centrifuged for 5 minutes at 5000 rpm. The supernatants were collected in separate 1.5 ml eppendorf tubes for further use and stored at −20° C., the cells were washed with 1× PBS, and the cell pellets were stored at −20° C. or immediately used for RNA isolation.

### ELISA

To detect the cytokine production following stimulation of PBMC, standard sandwich ELISAs (IFN-γ, TNF-α, GM-CSF, all purchased from Biolegend) were performed according to the manufacturer's instructions. The plate was read by Synergy H1 Hybrid reader from BioTek and the data recorded. The data were analyzed by Excel and GraphPad Prism.

### RT-PCR

RNA was isolated using the RNA Easy Plus Kit (Qiagen) according to the manufacturer's protocol. After isolation the RNA concentration and purity was determine using the Take 3 plate and the Synergy H1 Hybrid reader. The purity was also checked by 1% agarose gel. Reverse transcription PCR performed by the iScript cDNA Synthesis Kit (Biorad) according to the manufacturer's directions. The PCR was done with primers using proprietary sequences generated by Qiagen that were specific for IFN-γ (cat. #PPH00380C) and 18S (cat. #PPH05666E). Primers for Vα24 (Va24_CCY9XUZ, cat. #4400294) were purchased from Applied Biosciences by Life Technologies. For PCR, the HotStarTaq Plus Master Mix kit (Qiagen) was used and the PCR protocol included 35 cycles. For each sample: 10 μl master mix, 2 μl Coral load, 6 μl Nuclease free water, 1 μl cDNA and 1 μl primer set were used.

Real-Time Quantitative PCR (qPCR)

To measure the induction of IFN-γ mRNA in the stimulated PBMC qPCR was performed. The ABI Sybr Green master mix and HotStarTaq master mix kit from QIAGEN was used. qPCR was performed using primers specific for 185, Vα24 and IFN-γ, as described above. The total volume of the reaction mix was 20 μl and consisted of 10 μl master mix, 1 μl primer mix(3 μM), 5 μl H₂O and 4 μl cDNA (diluted 1:10). The Applied Biosystems 7500 Fast Real Time PCR system was used. The C_{T} values were collected and the fold increase calculated as follows: n-fold increase in IFN-γ mRNA=2^{[-(CTsample-CT 18S rRNA)-(CTempty beads-CT 18S rRNA)]}where CT is the threshold cycle.

### Antibodies and Flow Cytometry

Data were acquired with a BD LSR II Flow Cytometer (BD Biosciences) and analyzed with FCS Express V3 (De Novo Software, Los Angeles, Calif.). Doublets were excluded with FSC-A and FSC-H linearity. Human antibodies were as follows: anti-TCR Vα24-Jα18 (clone 6B11), anti-CD3 (clone UCHT1)—all purchased from BD Biosciences, anti-TCR Vα24 (clone C15) and anti-TCR Vβ11 (Beckman Coulter), and CD1d tetramers loaded with PBS57, an analog of α-GalCer (National Institutes of Health Tetramer Core Facility, Atlanta, Ga.).

### Statistical Analysis

An unpaired two-tailed Student t test was performed by Prism software (version 5.02 for Windows; GraphPad) to compare healthy donors to cancer patients. A p value <0.05 was considered significant. The error bars in the bar graphs show the S.E.M.

### Results

### Circulating NKT Cells Levels are Low

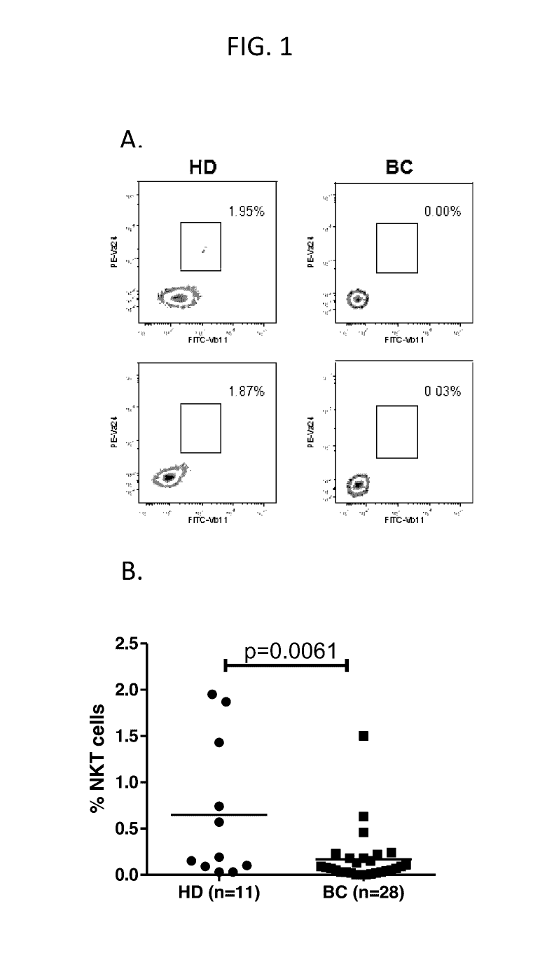

Despite the importance of NKT cells in regulating immune responses, their low frequency significantly restricts their potential for clinical application. As a precursor to developing a method to rapidly assess their function, we first examined the percentage of NKT cells in healthy donors and newly diagnosed breast cancer patients prior to treatment or surgery (FIG. 1A). As shown in FIG. 1B, the percentage of circulating NKT cells within the lymphocyte population was 0.17±0.06 in breast cancer patients (N=28) compared to 0.65±0.23 in healthy donors (N=11). NKT cells were significantly reduced in breast cancer patients compared to healthy donors (p=0.0016).

### Human T Cells Produce Cytokine Within Hours Following Stimulation

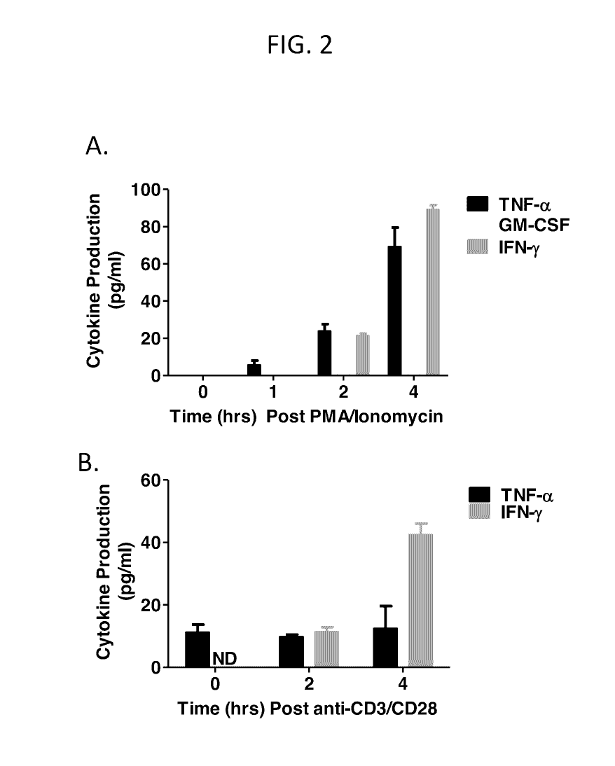

Given the low frequency of NKT cells within the peripheral blood, the goal of this study was to develop a sensitive assay in order to assess NKT cell function. First, we conducted time course studies to determine the kinetics of cytokine production. PBMC were stimulated for 0, 1, 2 and 4 hours with PMA and ionomycin. As shown in FIG. 2A, cytokine production could be detected by ELISA within four hours following stimulation. It was found that non-specific stimulation with PMA and ionomycin resulted in the rapid production of TNF-α, GM-CSF, IFN-γ and IL-17A (data not shown). Therefore, we next assessed T cell activation by stimulating the PBMC with anti-CD3/CD28 microbeads (FIG. 2B). We found that TNF-α and IFN-γ were quickly and reproducibly induced following stimulation. Notably, it was found that IFN-γ was consistently produced at high levels and could be used to measure T cell function in our studies.

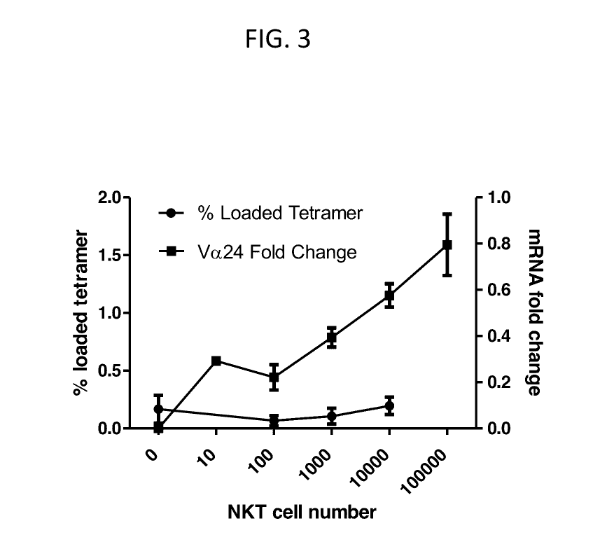

We conducted studies to determine the sensitivity of qPCR compared to flow cytometry for examining the NKT cell population. In order to assess the sensitivity of each assay, we sought to determine the lowest percentage of NKT cells that could be accurately measured by qPCR. To do these studies, purified NKT cells were serially diluted into a million PBMC and IFN-γ and Vα24 transcripts were measured by qPCR. We were able to detect a clear linear relationship between 100-10⁴ NKT cells (Vα24Jα18) and IFN-γ mRNA (data not shown). We also compared the Vα24 transcripts by qPCR results to α-GalCer tetramer⁺ NKT cells by flow cytometry (FIG. 3). We were able to detect low frequencies of NKT cells by flow cytometry; however, we found that qPCR was extremely sensitive in detecting increases in Vα24 transcripts.

aAPC-qPCR can be Used to Assess NKT Cell Function

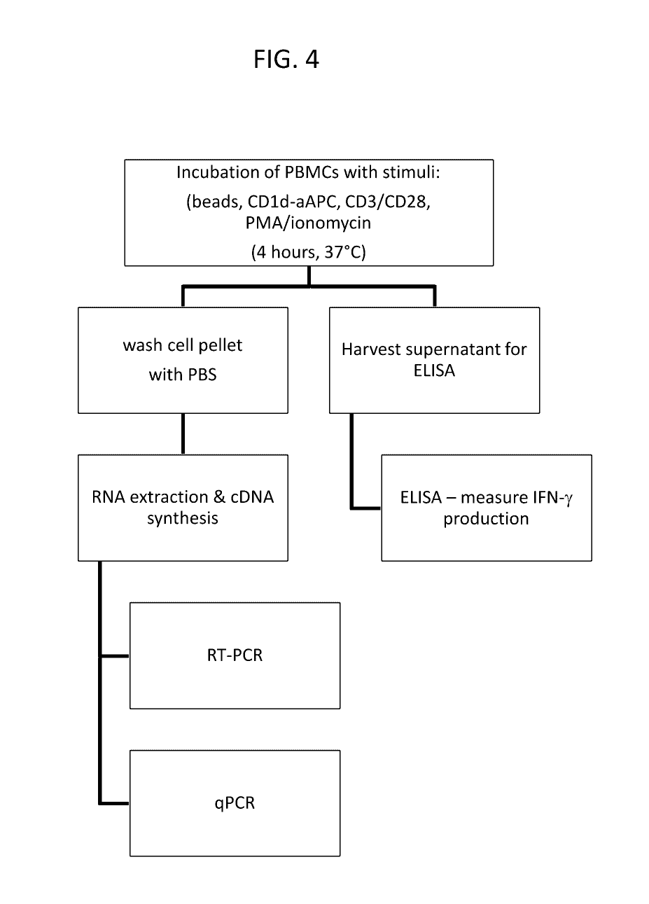

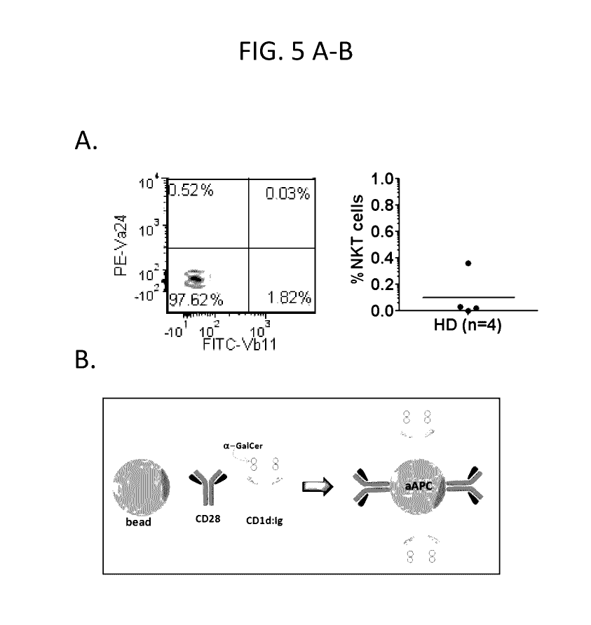

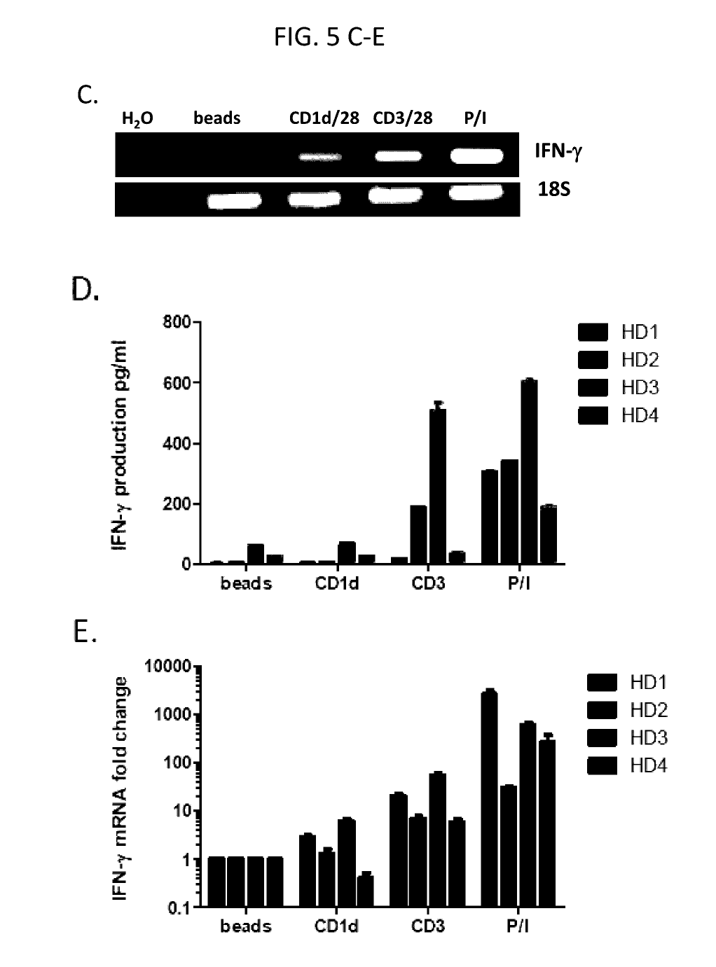

Studies from our laboratory and others have shown that α-GalCer artificial antigen presenting cells (aAPC) can be used to activate NKT cells (Shiratsuchi, T., Schneck, J., Kawamura, A. and Tsuji, M., 2009, Human CD1 dimeric proteins as indispensable tools for research on CD1-binding lipids and CD1-restricted T cells. J Immunol Methods 345, 49-59; Webb, T. J., Bieler, J. G., Schneck, J. P. and Oelke, M., 2009, Ex vivo induction and expansion of natural killer T cells by CD1d1-Ig coated artificial antigen presenting cells. J Immunol Methods 346, 38-44; Sun, W., Subrahmanyam, P. B., East, J. E. and Webb, T. J., 2012, Connecting the dots: artificial antigen presenting cell-mediated modulation of natural killer T cells. J Interferon Cytokine Res 32, 505-16). Thus, after our initial studies defined the optimal incubation time for assessing T cell activation, PBMC of healthy donors were incubated for four hours with various stimuli (see schematic of experimental design in FIG. 4). The percentage of NKT cells from each donor is shown in FIG. 4A. Healthy donors with relatively low circulating numbers of NKT cells were chosen to assess the sensitivity of this assay. To specifically activate NKT cells, α-GalCer loaded aAPC were used to stimulate the PBMC, empty beads were used as a negative control, anti-CD3/CD28 microbeads were used to measure total T cell function, and PMA/ionomycin was used as a positive control. The induction of IFN-γ was assessed as an indication of T cell activation and was measured by ELISA, conventional RT-PCR, and qPCR (FIGS. 5B-E). Following activation, there was a clear induction of IFN-γ production in NKT cells following α-GalCer-aAPC stimulation by RT-PCR and qPCR. Data shown in FIG. 5C is from Donor 1. As shown in the ELISA data (FIG. 5D), IFN-γ induction was not as profound in all donors and an extremely sensitive method is required to specifically detect NKT cell activation. The qPCR-aAPC method was able to detect NKT cell activation in 3 out of 4 donors, as shown in FIG. 5, whereas NKT cell activation was undetectable by ELISA. We were able to detect NKT cell responses, as assessed by IFN-γ induction, in all healthy donors with high percentages of circulating NKT cells (>0.5%; data not shown).

### NKT Cell Function can be Rapidly Assessed in Breast Cancer Patients

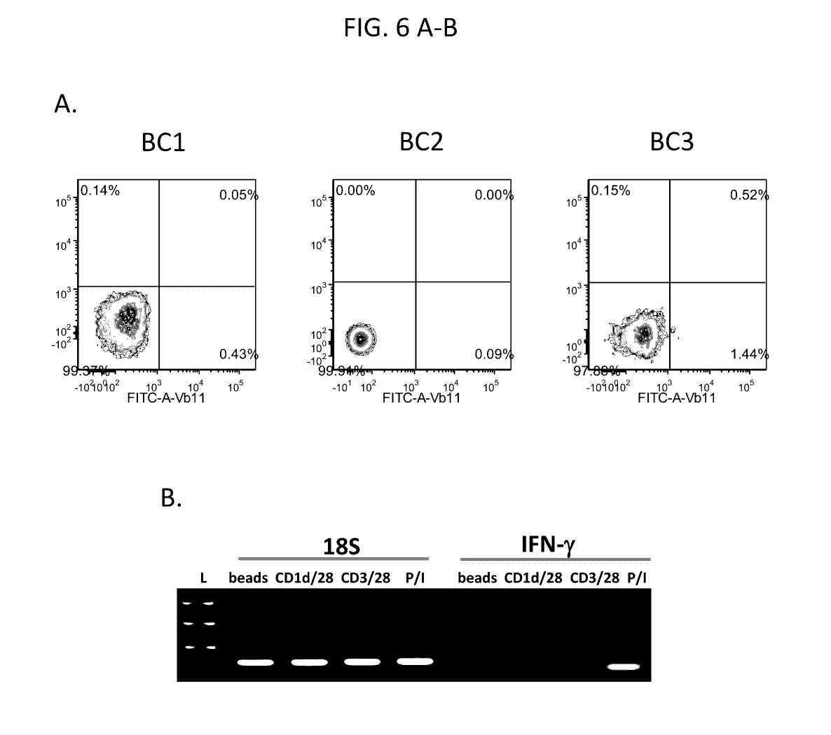

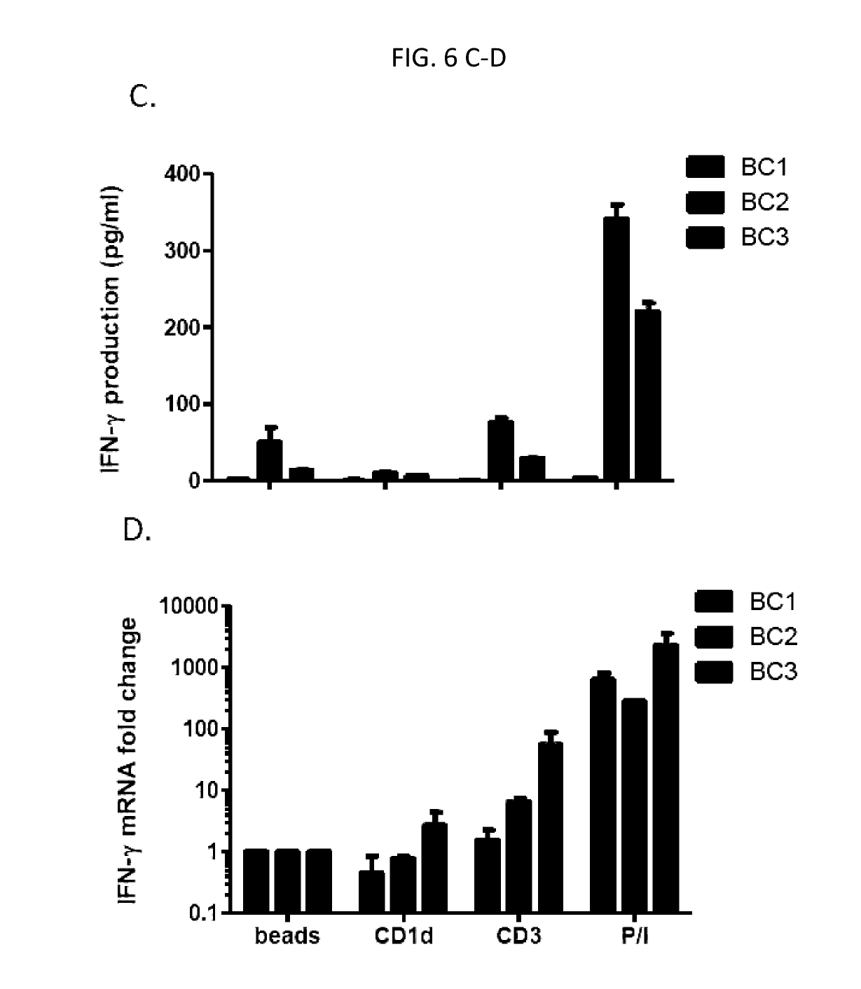

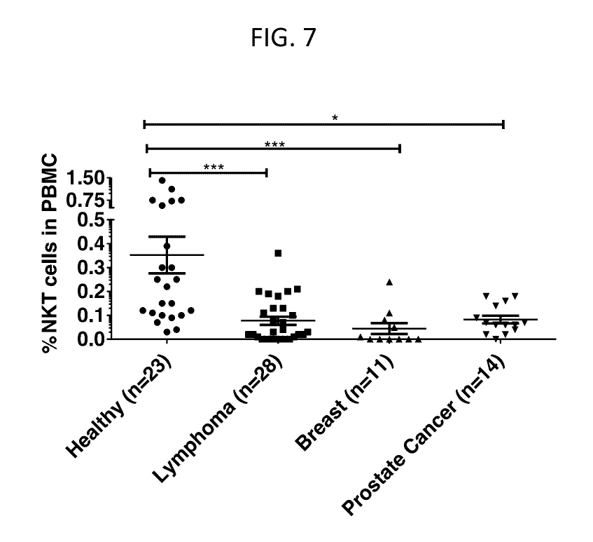

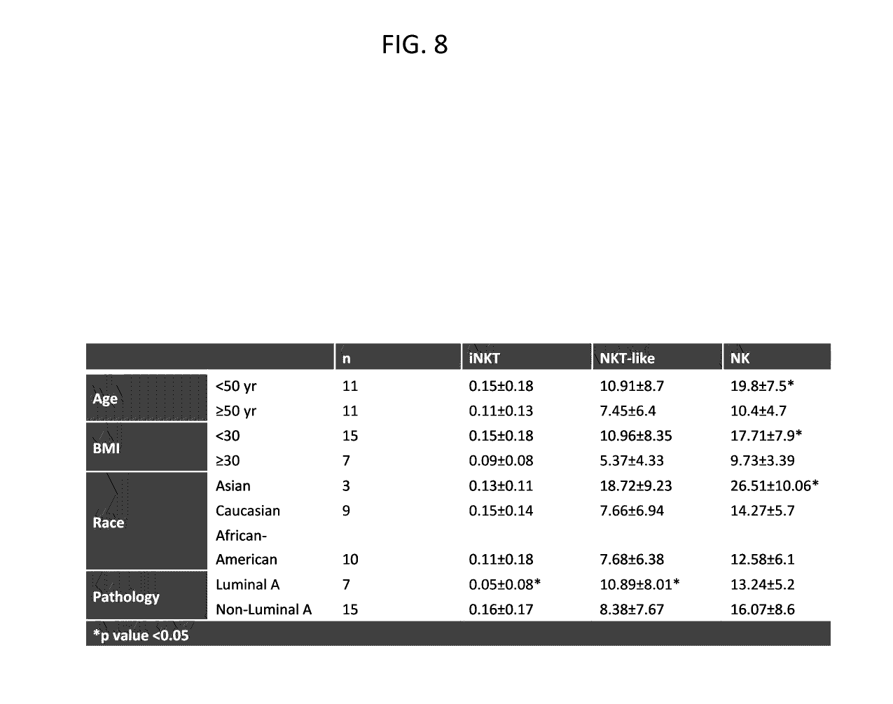

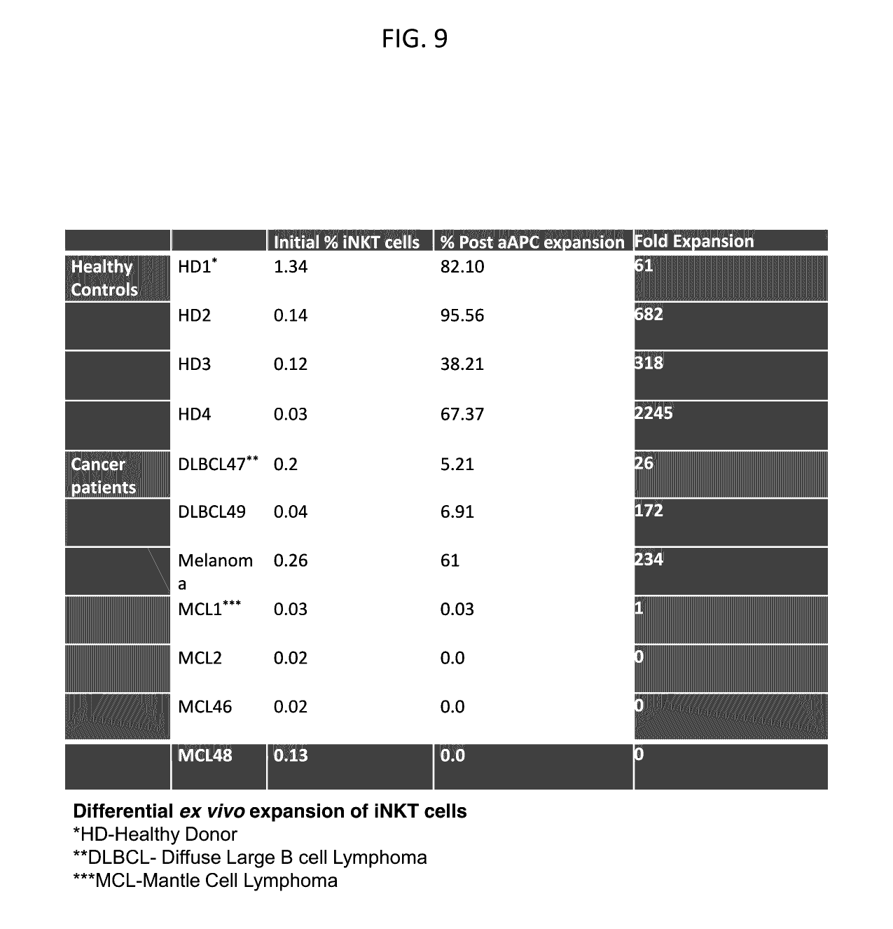

We have shown that aAPC-qPCR can be used to measure circulating NKT cell responses in the PBMC from healthy donors, but NKT cells have been reported to be reduced in number and function in cancer patients. As shown in FIGS. 1 and 6A, the circulating percentage of NKT cells in the peripheral blood of breast cancer is low. Thus, we next sought to determine if this qPCR-aAPC method can be used as a tool to determine baseline NKT cell function in breast cancer patients. In these studies, PBMC from breast cancer patients were stimulated with α-GalCer loaded aAPC and then functional studies were performed. We found it difficult to assess NKT cell activation by both classic RT-PCR (FIG. 6B) and ELISA (FIG. 6C); however, we were able to detect NKT cell activation by qPCR (FIG. 6D). Taken together our data shown that α-GalCer loaded aAPC can be used in combination with qPCR to investigate baseline NKT cell function in healthy donors and cancer patients.

### Discussion

NKT cells comprise a rare, but important subset of T cells that are activated following the recognition cognate lipid antigen presented in the context of CD1D (Godfrey, D. I., MacDonald, H. R., Kronenberg, M., Smyth, M. J. and Van Kaer, L., 2004, NKT cells: what's in a name? Nat Rev Immunol 4, 231-7). Following activation, NKT cells can directly lyse tumors and rapidly produce a plethora of cytokines (Fowlkes, B. J., Kruisbeek, A. M., Ton-That, H., Weston, M. A., Coligan, J. E., Schwartz, R. H. and Pardoll, D. M., 1987, A novel population of T-cell receptor ab-bearing thymocytes which predominantly expresses a single Vb gene family Nature 329, 251-4; Berzins, S. P., Smyth, M. J. and Baxter, A. G., 2011, Presumed guilty: natural killer T cell defects and human disease. Nat Rev Immunol 11, 131-42). Thus, NKT cell are considered to be important for immune surveillance (Prigozy, T. I., Naidenko, O., Qasba, P., Elewaut, D., Brossay, L., Khurana, A., Natori, T., Koezuka, Y., Kulkarni, A. and Kronenberg, M., 2001, Glycolipid antigen presentation by CD1d molecules. Science 291, 664-667). NKT cells have been demonstrated to play a role in autoimmune disease (Illes, Z., Kondo, T., Newcombe, J., Oka, N., Tabira, T. and Yamamura, T., 2000, Differential expression of NK T cell V alpha 24J alpha Q invariant TCR chain in the lesions of multiple sclerosis and chronic inflammatory demyelinating polyneuropathy. J Immunol 164, 4375-81), tumor surveillance (Terabe, M. and Berzofsky, J. A., 2008, The role of NKT cells in tumor immunity. Advances in cancer research 101, 277-348; Swann, J. B., Uldrich, A. P., van Dommelen, S., Sharkey, J., Murray, W. K., Godfrey, D. I. and Smyth, M. J., 2009, Type I natural killer T cells suppress tumors caused by p53 loss in mice. Blood 113, 6382-5), hematological cancers (Neparidze, N. and Dhodapkar, M. V., 2009, Harnessing CD1d-restricted T cells toward antitumor immunity in humans. Annals of the New York Academy of Sciences 1174, 61-7), infectious disease (Prigozy, T. I., Naidenko, O., Qasba, P., Elewaut, D., Brossay, L., Khurana, A., Natori, T., Koezuka, Y., Kulkarni, A. and Kronenberg, M., 2001, Glycolipid antigen presentation by CD1d molecules. Science 291, 664-667), and inflammatory conditions such as ischemia reperfusion injury (Kinjo, Y., Wu, D., Kim, G., Xing, G. W., Poles, M. A., Ho, D. D., Tsuji, M., Kawahara, K., Wong, C. H. and Kronenberg, M., 2005, Recognition of bacterial glycosphingolipids by natural killer T cells. Nature 434, 520-5).

Despite the importance of NKT cells in regulating immune responses, their low number diminishes their potential for clinical application. Previous studies have shown that circulating NKT cell numbers are reduced in cancer patients (Kawano, T., Nakayama, T., Kamada, N., Kaneko, Y., Harada, M., Ogura, N., Akutsu, Y., Motohashi, S., Iizasa, T., Endo, H., Fujisawa, T., Shinkai, H. and Taniguchi, M., 1999, Antitumor cytotoxicity mediated by ligand-activated human V alpha24 NKT cells. Cancer Res 59, 5102-5; Tahir, S. M., Cheng, O., Shaulov, A., Koezuka, Y., Bubley, G. J., Wilson, S. B., Balk, S. P. and Exley, M. A., 2001, Loss of IFN-gamma production by invariant NK T cells in advanced cancer. J Immunol 167, 4046-50; Giaccone, G., Punt, C. J., Ando, Y., Ruijter, R., Nishi, N., Peters, M., Von Blomberg, B. M., Scheper, R. J., Van Der Vliet, H. J., Van Den Eertwegh, A. J., Roelvink, M., Beijnen, J., Zwierzina, H. and Pinedo, H. M., 2002, A Phase I study of the natural killer T-cell ligand α-galactosylceramide (KRN7000) in patients with solid tumors. Clin Cancer Res 8, 3702-9; Motohashi, S., Kobayashi, S., Ito, T., Magara, K. K., Mikuni, O., Kamada, N., Iizasa, T., Nakayama, T., Fujisawa, T. and Taniguchi, M., 2002, Preserved IFN-alpha production of circulating Valpha24 NKT cells in primary lung cancer patients. Int J Cancer 102, 159-65; Crough, T., Purdie, D. M., Okai, M., Maksoud, A., Nieda, M. and Nicol, A. J., 2004, Modulation of human Valpha24(+)Vbeta11(+) NKT cells by age, malignancy and conventional anticancer therapies. Br J Cancer 91, 1880-6; Nieda, M., Okai, M., Tazbirkova, A., Lin, H., Yamaura, A., Ide, K., Abraham, R., Juji, T., Macfarlane, D. J. and Nicol, A. J., 2004, Therapeutic activation of Valpha24+Vbeta11+NKT cells in human subjects results in highly coordinated secondary activation of acquired and innate immunity. Blood 103, 383-9; Chang, D. H., Osman, K., Connolly, J., Kukreja, A., Krasovsky, J., Pack, M., Hutchinson, A., Geller, M., Liu, N., Annable, R., Shay, J., Kirchhoff, K., Nishi, N., Ando, Y., Hayashi, K., Hassoun, H., Steinman, R. M. and Dhodapkar, M. V., 2005, Sustained expansion of NKT cells and antigen-specific T cells after injection of alpha-galactosyl-ceramide loaded mature dendritic cells in cancer patients. J Exp Med 201, 1503-17; Moiling, J. W., Kolgen, W., van der Vliet, H. J., Boomsma, M. F., Kruizenga, H., Smorenburg, C. H., Molenkamp, B. G., Langendijk, J. A., Leemans, C. R., von Blomberg, B. M., Scheper, R. J. and van den Eertwegh, A. J., 2005, Peripheral blood IFN-gamma-secreting Valpha24+Vbeta11+NKT cell numbers are decreased in cancer patients independent of tumor type or tumor load. Int J Cancer 116, 87-93). While NKT cells are low in cancer patients, their strong anti-tumor functions have lead several groups to attempt to activate NKT cells in cancer patients (Nieda, M., Okai, M., Tazbirkova, A., Lin, H., Yamaura, A., Ide, K., Abraham, R., Juji, T., Macfarlane, D. J. and Nicol, A. J., 2004, Therapeutic activation of Valpha24+Vbeta11+NKT cells in human subjects results in highly coordinated secondary activation of acquired and innate immunity. Blood 103, 383-9; Ishikawa, A., Motohashi, S., Ishikawa, E., Fuchida, H., Higashino, K., Otsuji, M., Iizasa, T., Nakayama, T., Taniguchi, M. and Fujisawa, T., 2005, A phase I study of alpha-galactosylceramide (KRN7000)-pulsed dendritic cells in patients with advanced and recurrent non-small cell lung cancer. Clin Cancer Res 11, 1910-7; Uchida, T., Horiguchi, S., Tanaka, Y., Yamamoto, H., Kunii, N., Motohashi, S., Taniguchi, M., Nakayama, T. and Okamoto, Y., 2008, Phase I study of alpha-galactosylceramide-pulsed antigen presenting cells administration to the nasal submucosa in unresectable or recurrent head and neck cancer. Cancer Immunol Immunother 57, 337-45). These studies showed promise for NKT cell based immunotherapeutic strategies, because it was found that cancer patients that received α-GalCer-pulsed dendritic cells had enhanced NK and CD8+ T cell responses. However, the best responses were observed in patients with detectable baseline levels of NKT cells. Thus, in the current study we have developed a novel method to assess baseline NKT function in healthy donors and breast cancer patients using aAPC in combination with qPCR, in order to help to determine which patients may benefit most from NKT cell-based therapies.

As expected, we found that induction of NKT cells in breast cancer patients was reduced compared to healthy donors. Specifically, there was a modest 3 fold increase in IFN-γ by qPCR in one of the breast cancer patients following stimulation with α-GalCer-loaded aAPC, compared to cells incubated with the control beads. Notably, the lymphocytes from healthy donors and breast cancer patients were activated by anti-CD3/CD28 and PMA/ionomycin and this could be observed by ELISA as well as by conventional RT-PCR. These data highlight the need for a sensitive method for NKT cell activation because we were able to detect NKT cell activation in very few patient samples. Likewise, our data show that the mRNA fold change is much higher in healthy donors, compared to breast cancer patients for each type of stimulation.

It has been recently reported by Schneiders, et al. that levels of NKT cells were strong predictors of clinical outcome in patients with HNSCC treated with curative-intent radiotherapy (Schneiders, F. L., de Bruin, R. C., van den Eertwegh, A. J., Scheper, R. J., Leemans, C. R., Brakenhoff, R. H., Langendijk, J. A., Verheul, H. M., de Gruijl, T. D., Moiling, J. W. and van der Vliet, H. J., 2012, Circulating invariant natural killer T-cell numbers predict outcome in head and neck squamous cell carcinoma: updated analysis with 10-year follow-up. J Clin Oncol 30, 567-70). In this study, the authors further highlight the prognostic potential of NKT cells since their group and others have established a direct relation between a low frequency of intratumoral NKT cells and poor prognosis (van der Vliet, H. J., Balk, S. P. and Exley, M. A., 2006, Natural killer T cell-based cancer immunotherapy. Clin Cancer Res 12, 5921-3). We have validated our method using fresh and frozen PBMCs and are currently working to optimize the conditions needed to assess NKT cell function using whole blood.

In summary, herein we show that NKT cells function can be rapidly assessed in vitro after stimulation with aAPC in healthy donors and breast cancer patients. Although activation levels were highly donor specific, we were able to detect a clear induction in IFN-γ mRNA following stimulation. Ongoing studies will help us to address the mechanisms by which NKT cells are numerically reduced and functionally impaired in breast cancer patients; however, this method has the potential to provide a better understanding of which patients may benefit from NKT cell-based immunotherapeutic strategies.

While there have been shown and described what are presently believed to be the preferred embodiments of the present invention, those skilled in the art will realize that other and further embodiments can be made without departing from the spirit and scope of the invention described in this application, and this application includes all such modifications that are within the intended scope of the claims set forth herein. All patents and publications mentioned and/or cited herein are incorporated by reference to the same extent as if each individual publication was specifically and individually indicated as having been incorporated by reference in its entirety.

What is claimed is:

1. A method of determining T cell function in a subject in need of immunotherapy comprising:
i) providing a blood sample comprising a population of T cells from the subject;
ii) activating the T cells in the sample; and
iii) assaying an expression level of one or more T cell activation markers using quantitative real time PCR (qPCR) after activating the T cells in the sample.

2. The method of claim 1, wherein the blood sample comprises isolated peripheral blood mononuclear cells (PBMC).

3. The method of any of claims 1-2, wherein the subject is afflicted with a disease.

4. The method of any of claims 1-2, wherein the subject has an infection.

5. The method of claim 4, wherein the disease is cancer.

6. The method of claim 5, wherein the cancer is selected from the group consisting of breast cancer; bladder cancer; lung cancer; prostate cancer;
thyroid cancer; leukaemia, lymphoma, CLL (chronic lymphocytic leukemia), CML (chronic myelocytic leukaemia), ALL (acute lymphoblastic leukaemia), AML (acute myelocytic leukaemia), PML (pro-myelocytic leukaemia), T-cell lymphoma, colon cancer; glioma; seminoma; liver cancer;
pancreatic cancer; bladder cancer; renal cancer; cervical cancer; testicular cancer; head and neck cancer; ovarian cancer; neuroblastoma and melanoma.

7. The method of claim 4, wherein the infection is a bacterial infection, a viral infection, or a parasitic infection.

8. The method of claim 7, wherein the infection is a bacterial infection from a bacteria selected from the group consisting of Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracia, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospria spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., and Rickettsia spp.

9. The method of claim 7, wherein the infection is a parasitic infection.

10. The method of claim 9, wherein the subject has a parasitic infection selected from the group consisting of amebiasis from Entamoeba histolytica, amebic meningoencephalitis from the genus Naegleria or Acanthamoeba, malaria from Plasmodium vivax, Plasmodium ovale, Plasmodium malariae, and Plasmodium falciparum, leishmaniasis from protozoa Leishmania donovani, Leishmania infantum, Leishmania chagasi, Leishmania tropica, Leishmania major, Leishmania aethiopica, Leishmania mexicana, and Leishmania braziliensis, Chagas' disease from the protozoan Trypanosoma cruzi, sleeping sickness from Trypanosoma brucei, Trypanosoma gambiense, and Trypanosoma rhodesiense, toxoplasmosis from Toxoplasma gondii, giardiasis from Giardia lamblia, cryptosporidiosis from Cryptosporidium parvum, trichomoniasis from Trichomonas vaginalis, Trichomonas tenax, Trichomonas hominis, pneumocystis pneumonia from Pneumocystis carinii, bambesosis from Bambesia microti, Bambesia divergens, and Bambesia boris.

11. The method of claim 7, wherein the infection is a viral infection caused by any one of a member of the Adenoviridae family, a member of the Coronavirus family, a member of the Picornaviridae family, a member of the Herpesviridae family, a member of the Hepadnaviridae family, a member of the Flaviviridae family, a member of the Retroviridae family, a member of the Orthomyxoviridae family, a member of the Paramyxoviridae family, a member of the Papovaviridae family, a member of the Rhabdoviridae family, or a member of the Togaviridae family

12. The method of any of claims 1-11, wherein the T cells are Natural Killer T cells.

13. The method of any of claims 1-12, wherein the subject is a mammal

14. The method of any of claims 1-13, wherein the mammal is selected from the group consisting of a human, mouse, rat, guinea pig, cat, dog, horse, cow, sheep or pig.

15. The method of any of claims 1-14 wherein the T cells are activated by CD1d bound to ligand.

16. The method of claim 15, wherein the CD1d bound ligand is selected from the group consisting of C-glycosidific form of alpha-galactosylceramide (α-C-GalCer, alpha-galactosylceramide (α-GalCer), 12 carbon acyl form of galactosylceramide (β-GalCer (C12)), β-D-glucopyranosylceramide (β-GlcCer), 1,2diacyl-3-O-galactosyl-sn-glycerol (BbGL-II), diacylglycerol containing glycolipids (Glc-DAG-s2), Ganglioside (GD3), gangliotriaosylceramide (Gg3Cer), glycosylphosphatidylinositol (GPI), alpha-glucuronosylceramide (GSL-1), alpha-glucuronosylceramide (GSL-4), house dust extract+ovalbumin (HDE+OVA), isoglobotrihexosylceramide (iGb3), lipophosphoglycan (LPG), lyosphosphatidylcholine (LPC), alpha-galactosylceramide analog (OCH), phosphatidylcholine (PC), phosphatidylethanolamine (PE), phosphatidylinositol (PI), PI dimannoside (PIM4), phenyl pentamethyldihydrobenzofuran sulfonates (PPBF), sulfatide, phosphatidylserine (PS), threitolceramide and combinations thereof.

17. The method of any of claims 1-16, wherein the method further comprises assaying an expression level of one or more T cell activation markers using quantitative real time PCR (qPCR) from control T cells that have not been activated.

18. The method of any of claims 17, wherein the method comprises comparing the expression level of one or more T cell activation markers from the activated with the expression level of one or more T cell activation markers from control T cells.

19. The method of claim 17, wherein the activated T cells and the control T cells are from the same subject.

20. The method of any of claims 1-19, further comprising obtaining or assaying an expression level of one or more T cell activation markers using quantitative real time PCR (qPCR) from a control, healthy subject.

21. The method of any of claims 1-20, wherein the method further comprises comparing the expression level of one or more T cell activation markers from the activated T cells from the subject in need of immunotherapy with the expression level of one or more T cell activation markers from activated T cells from a control, healthy subject.

22. The method of any of claims 1-21, further comprising administering to the subject an effective amount of immunotherapy.

23. The method of any of claims 1-22, wherein if the expression level of the one or more T cell activation markers from the activated T cells from the subject in need of immunotherapy is at least 30% of the expression level of one or more T cell activation markers from activated T cells from a control, healthy subject, then the subject in need of immunotherapy is administered an effective amount of immunotherapy.

24. The method of any of claims 1-21, wherein the subject is not administered an immunotherapy when the expression level of the one or more T cell activation markers from the activated T cells from the subject in need of immunotherapy is below 10% of the expression level of one or more T cell activation markers from activated T cells from a control, healthy subject.

25. The method of any of claims 1-24, wherein PBMC are isolated using Ficoll density gradient.

26. The method of any of claims 1-25, wherein approximately 10⁶ PBMC cells are stimulated.

27. The method of any of claims 1-26, wherein the T cells are stimulated with CD1d bound to α-GalCer.

28. The method of any of claims 1-26, wherein the T cells are stimulated with an antigen presenting cell comprising CD1d.

29. The method of any of claims 1-26, wherein the T cells are stimulated with an antigen presenting cell comprising CD1d bound to α-GalCer.

30. The method of any of claims 1-26, wherein the T cells are stimulated with an artificial antigen presenting cell.

31. The method of any of claims 1-26, wherein the T cells are stimulated with an artificial antigen presenting cell comprising CD1d bound to α-GalCer.

32. The method of claim 30, wherein the artificial antigen presenting cell comprises a magnetic bead loaded with CD1d bound to ligand.

33. The method of claim 32, wherein the ligand is α-GalCer.

34. The method of any of claims 1-26, wherein the total T cells are stimulated with an antigen presenting cell comprising anti-CD3 and anti-CD28.

35. The method of claim 30, wherein the artificial antigen presenting cell comprises a magnetic bead loaded with anti-CD3 and anti-CD28.

36. The method of any of claims 1-35, wherein the T cells are stimulated from 2-8 hours at 37° C.

37. The method of any of claims 1-36, wherein the T cells are stimulated for about 4 hrs at 37° C.

38. The method of any of claims 1-37, wherein the one or more T cell activation markers is selected from the group consisting of IFN-γ, TNF-α, and GM-CSF.

39. The method of any of claims 1-38, wherein the one or more T cell activation markers is induced at least 5-fold over cells that have not been activated.

40. The method of any of claims 1-39, wherein the one or more T cell activation markers is induced at least 10-fold over cells that have not been activated.

41. A method of determining NKT cell function in a subject in need of immunotherapy comprising:
i) providing a blood sample comprising a population of T cells from the subject;
ii) activating the NKT cells in the sample with an artificial antigen presenting cell comprising CD1d bound to α-GalCer; and
iii) assaying an expression level of one or more T cell activation markers using quantitative real time PCR (qPCR) after activating the T cells in the sample.

42. A method of determining total T cell function in a subject in need of immunotherapy comprising:
i) providing a blood sample comprising a population of T cells from the subject;
ii) activating the total T cells in the sample with an artificial antigen presenting cell comprising anti-CD3 and anti-CD28; and
iii) assaying an expression level of one or more T cell activation markers using quantitative real time PCR (qPCR) after activating the T cells in the sample.

44. A method of determining NKT cell function in a subject comprising:
i) providing a blood sample comprising a population of T cells from the subject;
ii) activating the NKT cells in the sample; and
iii) assaying an expression level of one or more T cell activation markers using quantitative real time PCR (qPCR) after activating the T cells in the sample, wherein the subject has a disease wherein NKT cells are aberrantly activated.

45. The method of claim 44, wherein the subject has a disease selected from the group consisting of asthma, psoriasis, and atherosclerosis.
